(19) United States
(12) Patent Application Publication
(10) Pub. No.: US 2009/0215997 A1
(43) Pub. Date: Aug. 27, 2009

(54) SEPARATION METHOD

(75) Inventors: Ken SUGO, Saitama (JP); Tomohiko YOSHITAKE, Tokyo (JP); Tsuneo OKUYAMA, Kanagawa (JP)

Correspondence Address: GREENBLUM & BERNSTEIN, P.L.C., 1950 ROLAND CLARKE PLACE, RESTON, VA, 20191, US

(73) Assignees: Tsuneo OKUYAMA, Kanagawa (JP); HOYA CORPORATION, Tokyo (JP)

(21) Appl. No.: 12/388,701

(22) Filed: Feb. 19, 2009

(30) Foreign Application Priority Data
Feb. 22, 2008 (JP) 2008-042209

(51) Int. Cl. C07K 1/00

(52) U.S. Cl. 530/415

(57) ABSTRACT

A method of separating a fluorescent protein from a sample containing a plurality of proteins containing the fluorescent protein is provided. The method comprising: preparing a sample solution by adding the sample to a liquid; preparing an adsorption apparatus having a filling space for filling an adsorbent having a surface, wherein at least the surface of the adsorbent is constituted of a calcium phosphate-based compound and at least a part of the filling space is filled with the adsorbent; supplying the sample solution into the filling space of the adsorption apparatus so that the plurality of proteins are adsorbed by the adsorbent; supplying a phosphate elution buffer for eluting the fluorescent protein contained in the plurality of proteins from the adsorbent into the filling space of the adsorption apparatus to thereby obtain an eluant containing the fluorescent protein; and fractionating the eluant which is discharged from the filling space of the adsorption apparatus into a portion of the phosphate elution buffer containing the fluorescent protein and other portions thereof to thereby separate the fluorescent protein from the plurality of proteins. According to the present invention, it is possible to separate a large amount of the fluorescent protein from the sample containing the plurality of proteins containing the fluorescent protein with high purity by a simple operation.

### FIELD OF THE INVENTION

The present invention relates to a separation method, particularly to a method of separating a fluorescent protein from a sample containing a plurality of proteins.

### BACKGROUND ART

As a method of detecting an object to be detected with high sensitivity by utilizing an antigen-antibody reaction, an enzyme-linked immunosorbent assay (ELISA) or the like is used.

Such an enzyme-linked immunosorbent assay uses a reagent obtained by, for example, preparing an antibody that can be specifically bonded to an object to be detected (i.e., an antigen) and allowing a fluorescent material as a marker to be carried on (bound to) the antibody.

In recent years, the use of fluorescent proteins (Green Fluorescent Protein; GFP) derived from Aequorea coerulescens which is a kind of cnidarian as such fluorescent materials has been contemplated.

For example, a method of separating a fluorescent protein from threads constituting a silkworm cocoon by using a Ni-affinity column is disclosed in M. Tomita et al., Transgenic Res., 16, 449-465, 2007. The method is carried out by transferring a nucleic acid including a gene corresponding to the fluorescent protein to a nucleic acid of the silkworm to thereby express the fluorescent protein in the threads constituting the silkworm cocoon.

However, since an adsorbent (separating agent) used in the Ni-affinity column is constituted of resin materials as a main component thereof, the adsorbent filled into a lower portion of a filling space of the Ni-affinity column is deformed or destroyed under its own weight by repeating the use of the adsorbent. Therefore, there is a problem that clogging occurs in the lower portion of the filling space of the Ni-affinity column.

Such a problem becomes conspicuous in the case where a column size is scaled-up for separating a large amount of a fluorescent protein at a time.

Further, since Ni, which is a hazardous metal, is filled into the filling space of the Ni-affinity column, it is not preferred that the Ni-affinity column is used for separating the fluorescent protein from a viewpoint of an environment.

It is an object of the present invention to provide a separation method capable of separating a large amount of a fluorescent protein from a sample (sample solution) containing a plurality of proteins containing the fluorescent protein with high purity by a simple operation.

This object is achieved by the present inventions (1) to (14) described below.

(1) A method of separating a fluorescent protein from a sample containing a plurality of proteins containing the fluorescent protein is provided. The method comprises: preparing a sample solution by adding the sample to a liquid; preparing an adsorption apparatus having a filling space for filling an adsorbent having a surface, wherein at least the surface of the adsorbent is constituted of a calcium phosphate-based compound and at least a part of the filling space is filled with the adsorbent; supplying the sample solution into the filling space of the adsorption apparatus so that the plurality of proteins are adsorbed by the adsorbent; supplying a phosphate elution buffer for eluting the fluorescent protein contained in the plurality of proteins from the adsorbent into the filling space of the adsorption apparatus to thereby obtain an eluant containing the fluorescent protein; and fractionating the eluant which is discharged from the filling space of the adsorption apparatus into a portion of the phosphate elution buffer containing the fluorescent protein and other portions thereof to thereby separate the fluorescent protein from the plurality of proteins.

According to the method described above, it is possible to be capable of separating a large amount of the fluorescent protein from the sample (sample solution) containing the plurality of proteins containing the fluorescent protein with high purity by a simple operation.

(2) In the method described in the above-mentioned item (1), in the phosphate elution buffer supplying step, a pH of the phosphate elution buffer is in the range of 6 to 8.

This makes it possible to prevent the fluorescent protein to be separated from being alterated and prevent fluorescence property thereof from being changed. Additionally, it is possible to reliably prevent the adsorbent from being alterated (dissolution and the like) so that it is possible to prevent separation capacity of the adsorbent from being changed in the adsorption apparatus.

(3) In the method described in the above-mentioned item (1), in the phosphate elution buffer supplying step, a temperature of the phosphate elution buffer is in the range of 30 to 50° C.

This makes it possible to prevent the fluorescent protein to be separated from being alterated.

(4) In the method described in the above-mentioned item (1), in the phosphate elution buffer supplying step, a salt concentration of the phosphate elution buffer is 500 mM or lower.

According to the method described above, it is possible to prevent adverse affects from occurring to the fluorescent protein by metal ions contained in the phosphate elution buffer.

(5) In the method described in the above-mentioned item (1), in the phosphate elution buffer supplying step and the eluant fractionating step, a flow rate of the phosphate elution buffer flowing in the filling space of the adsorption apparatus is in the range of 0.1 to 10 mL/min.

According to the method described above, it is possible to reliably separate a target fluorescent protein without long time to be needed to separation operations. That is to say, the fluorescent protein having high purity can be obtained.

(6) In the method described in the above-mentioned item (1), the fluorescent protein is at least one of a fluorescent protein derived from a cnidarian and an altered body thereof.

The method described above can be applied to a method of separating various kinds of fluorescent proteins from a sample. In particular, the method described above can be applied to a method of separating the fluorescent protein derived from the cnidarian and/or the altered body thereof from a sample.

(7) In the method described in the above-mentioned item (1), the altered body is obtained by adding at least one of histidine, lysine, and arginine to the fluorescent protein derived from the cnidarian.

In various kinds of amino acids, histidine, lysine, and arginine have high affinity to metal ions. Therefore, if the altered body of the fluorescent protein is produced by adding at least one of histidine, lysine, and arginine to a natural fluorescent protein, it becomes possible to collect the fluorescent protein with a high yield.

(8) In the method described in the above-mentioned item (1), the fluorescent protein is expressed in threads constituting a silkworm cocoon by transferring a nucleic acid including a gene corresponding to the fluorescent protein to a nucleic acid of the silkworm.

According to the method described above, it is possible to obtain a fluorescent protein having a simple structure. Therefore, it is possible to prevent adsorption property of the fluorescent protein to the adsorbent from being changed. That is, the method described above according to the present invention is optimum for separating such a fluorescent protein from the sample solution.

(9) In the method described in the above-mentioned item (1), the calcium phosphate-based compound is constituted of hydroxyapatite as a main component thereof.

Since hydroxyapatite is a substance similar to components of living body, it is possible to reliably prevent such a fluorescent protein from being altered (deactivated) when separating the fluorescent protein from the sample. Further, by changing a salt concentration of the phosphate elution buffer as an eluate, the method described above has an advantage that the fluorescent protein can be easily desorbed from the adsorbent to the phosphate elution buffer to obtain the eluant.

(10) In the method described in the above-mentioned item (9), the hydroxyapatite has hydroxyl groups, and the hydroxyapatite is reacted with hydrogen fluoride molecules having fluorine atoms to obtain a fluoroapatite, wherein at least one of the hydroxyl groups of the hydroxyapatite is substituted by the fluorine atoms of the hydrogen fluoride molecules.

Hydroxyapatite of which hydroxyl groups are substituted by the fluorine atoms of the hydrogen fluoride molecules, that is, fluoroapatite has the fluorine atoms (fluorine ions) in a chemical structure thereof. Therefore, it is possible to prevent calcium atoms (calcium ions) from being eliminated from fluoroapatite.

(11) In the method described in the above-mentioned item (10), the fluoroapatite is produced by preparing a slurry containing the hydroxyapatite, preparing a hydrogen fluoride-containing solution containing the hydrogen fluoride molecules, mixing the slurry and the hydrogen fluoride-containing solution to obtain a mixture thereof, and reacting the hydroxyapatite contained in the slurry and the hydrogen fluoride molecules contained in the hydrogen fluoride-containing solution in the mixture to thereby substitute the at least one of the hydroxyl groups of the hydroxyapatite to the fluorine atoms of the hydrogen fluoride molecules.

According to the method described above, since the hydrogen fluoride molecules are used as a fluorine source, it is possible to obtain fluoroapatite having high crystallinity in which no impurity is contained or an impurity is contained at a very low level.

(12) In the method described in the above-mentioned item (11), a pH of the mixture is in the range of 2.5 to 5.0.

According to the method described above, it is possible to obtain hydroxyapatite of which hydroxyl groups are substituted by the fluorine atoms of the hydrogen fluoride molecules, that is, fluoroapatite having high crystallinity.

(13) In the method described in the above-mentioned item (10), the fluoroapatite is produced by preparing a first liquid containing a calcium-based compound containing calcium, a second liquid containing the hydrogen fluoride and a third liquid containing phosphoric acid, respectively, and thereafter obtaining a first mixture by mixing the first liquid, the second liquid and the third liquid, and then reacting the calcium-based compound, the hydrogen fluoride and the phosphoric acid in the first mixture.

According to the method described above, since the hydrogen fluoride molecules are used as a fluorine source, it is possible to obtain hydroxyapatite of which hydroxyl groups are substituted by the fluorine atoms of the hydrogen fluoride molecules, that is, fluoroapatite having high crystallinity, in which no impurity is contained or an impurity is contained at a very low level.

(14) In the method described in the above-mentioned item (13), the first mixture obtaining step is carried out by mixing the second liquid and the third liquid to obtain a second mixture and thereafter mixing the second mixture with the first liquid.

This makes it possible to uniformly mix the second liquid and the third liquid with the first liquid to thereby produce fluoroapatite. Further, the hydroxyl groups of hydroxyapatite can be uniformly substituted by the fluorine atoms of the hydrogen fluoride molecules. Furthermore, it is also possible to reliably prevent or suppress a by-product such as calcium fluoride from being produced in the second mixture.

According to the present invention, it is possible to separate a large amount of the fluorescent protein from the sample (sample solution) containing the plurality of proteins containing the fluorescent protein with high purity by a simple operation.

By appropriately setting separating conditions such as the salt concentration or the flow rate of the phosphate elution buffer, though depending on a kind of fluorescent protein to be separated, it is possible to improve purity of the fluorescent protein to be separated and purified.

### BRIEF DESCRIPTION OF THE DRAWINGS

FIG. 1 is a sectional view which shows one example of an adsorption apparatus to be used in the present invention.

FIG. 2 shows an absorbance curve which are measured when a fluorescent protein contained in a sample solution are separated by using the adsorption apparatus.

### BEST MODE FOR CARRYING OUT THE INVENTION

Hereinbelow, a separation method according to the present invention will be described in detail based on a preferred embodiment shown in the accompanying drawings.

First, prior to the description of the separation method according to the present invention, one example of an adsorption apparatus (separation apparatus) to be used in the present invention will be described.

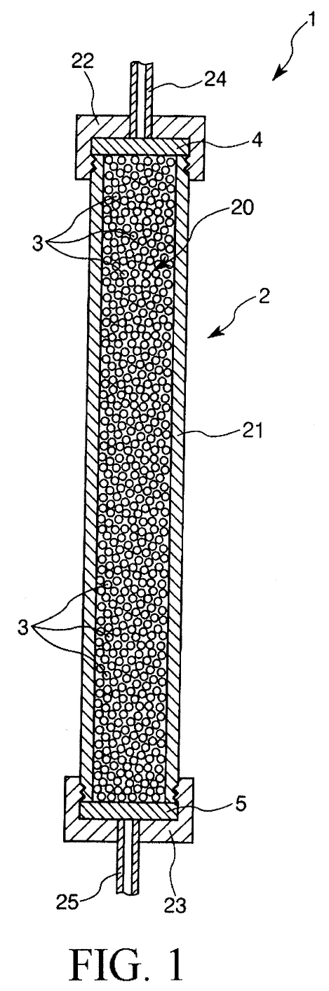

FIG. 1 is a sectional view which shows one example of an adsorption apparatus to be used in the present invention. It is to be noted that in the following description, the upper side and the lower side in FIG. 1 will be referred to as “inflow side” and “outflow side”, respectively.

More specifically, the inflow side means a side from which liquids such as a sample solution (i.e., a liquid containing a sample) and a phosphate elution buffer (i.e., an eluate) are supplied into the adsorption apparatus to separate (purify) a target fluorescent protein, and the outflow side means a side located on the opposite side from the inflow side, that is, a side through which the liquids described above discharge out of the adsorption apparatus.

Hereinafter, the description will be made on a case that a fluorescent protein derived from a cnidarian and/or an altered body thereof are/is used as the fluorescent protein to be separated by using the adsorption apparatus as a representative. The description will be also made on a case that the fluorescent protein is separated from the sample solution containing a plurality of proteins by using the adsorption apparatus as a representative.

In this regard, the altered body of the fluorescent protein derived from the cnidarian means a protein having fluorescing property (keeping fluorescence) and an amino-acid sequence in which one or more amino acids included in an amino-acid sequence of a fluorescent protein derived from a natural cnidarian are lost, and/or substituted by another amino acid, and/or the other amino acid is added to the one or more amino acids.

A fluorescent protein (Green Fluorescent Protein: GFP) which is possessed by Aequorea coerulescens which is a kind of cnidarian glows a fluorescent green. However, by changing a part of amino acids included in the amino-acid sequence of the fluorescent protein, it is known that a fluorescent proteins which glow a fluorescent red, a fluorescent yellow, fluorescent cyan, and the like are obtained. Examples of such fluorescent proteins include Red Fluorescent Protein (RFP), Yellow Fluorescent Protein (YFP), and Cyan Fluorescent Protein (CFP).

In this regard, it is to be noted that the fluorescent protein derived from Aequorea coerulescens can be extracted by using a genetic recombination technology disclosed in US-A-2008-0301823. That is, the fluorescent protein is produced in threads constituting a silkworm cocoon by using the genetic recombination technology, and then can be extracted by dipping the threads into an aqueous solution. Further, it is to be noted that an altered body of the fluorescent protein derived from Aequorea coerulescens can also be obtained by using them in combination with the genetic recombination technology disclosed in US-A-2008-0301823 and a general genetic recombination technology.

Hereinafter, the fluorescent protein derived from the cnidarian and the altered body thereof are simply and collectively referred to as “fluorescent protein derived from the cnidarian”.

The adsorption apparatus 1 shown in FIG. 1, which is used for separating the fluorescent protein derived from the cnidarian from the sample solution, includes a column 2, a granular adsorbent (filler) 3, and two filter members 4 and 5.

The column 2 is constituted from a column main body 21 and caps 22 and 23 to be attached to the inflow-side end and outflow-side end of the column main body 21, respectively.

The column main body 21 is formed from, for example, a cylindrical member. Examples of a constituent material of each of the parts (members) constituting the column 2 including the column main body 21 include various glass materials, various resin materials, various metal materials, and various ceramic materials and the like.

An opening of the column main body 21 provided on its inflow side is covered with the filter member 4, and in this state, the cap 22 is threadedly mounted on the inflow-side end of the column main body 21. Likewise, an opening of the column main body 21 provided on its outflow side is covered with the filter member 5, and in this state, the cap 23 is threadedly mounted on the outflow-side end of the column main body 21.

The column 2 having such a structure as described above has an adsorbent filling space 20 which is defined by the column main body 21 and the filter members 4 and 5, and at least a part of the adsorbent filling space 20 is filled with the adsorbent 3 (in this embodiment, almost the entire of the adsorbent filling space 20 is filled with the adsorbent 3).

A volumetric capacity of the adsorbent filling space 20 is appropriately set depending on the volume of a sample solution to be used. Such a volumetric capacity is not particularly limited, but is preferably in the range of about 0.1 to 100 mL, and more preferably in the range of about 1 to 50 mL per 1 mL of the sample solution.

By setting a size of the adsorbent filling space 20 to a value within the above range and by setting a size of the adsorbent 3 (which will be described later) to a value within a range as will be described later, it is possible to reliably and mutually separate the fluorescent protein derived from the cnidarian from contaminating proteins (foreign substances) other than the fluorescent protein derived from the cnidarian contained in the sample solution.

In this regard, it is to be noted that examples of the contaminating proteins contained in the sample solution include the following proteins.

First, a fluorescent protein is expressed in threads constituting a silkworm cocoon by transferring a nucleic acid including a gene corresponding to the fluorescent protein to a nucleic acid of the silkworm. Thereafter, the expressed fluorescent protein is extract (dissolved) in an aqueous solution to obtain an extraction solution which is used as the sample solution. In this case, examples of the contaminating proteins include proteins other than the fluorescent protein derived from the threads constituting the silkworm cocoon, which are extracted in the sample solution in the same manner as described above.

Further, liquid-tightness between the column main body 21 and the caps 22 and 23 is ensured by attaching the caps 22 and 23 to the openings of the column main body 21.

An inlet pipe 24 is liquid-tightly fixed to the cap 22 at substantially the center thereof, and an outlet pipe 25 is also liquid-tightly fixed to the cap 23 at substantially the center thereof. The liquids described above are supplied to the adsorbent filling space 20 through the inlet pipe 24 and the filter member 4. The liquids supplied to the adsorbent filling space 20 pass through gaps between particles of the adsorbent 3 and then discharge out of the column 2 through the filter member 5 and the outlet pipe 25. At this time, the fluorescent protein derived from the cnidarian and the contaminating proteins contained in the sample solution (sample) are separated from each other based on a difference in degree of adsorption of each of the fluorescent protein derived from the cnidarian and the contaminating proteins to the adsorbent 3 and a difference in degree of affinity of each of the fluorescent protein derived from the cnidarian and the contaminating proteins to a phosphate elution buffer.

Each of the filter members 4 and 5 has a function of preventing the adsorbent 3 from discharging out of the adsorbent filling space 20. Further, each of the filter members 4 and 5 is formed of a nonwoven fabric, a foam (a sponge-like porous body having communicating pores), a woven fabric, a mesh or the like, which is made of a synthetic resin such as polyurethane, polyvinyl alcohol, polypropylene, polyetherpolyamide, polyethylene terephthalate, or polybutylene terephthalate.

At least the surface of the adsorbent 3 is constituted of a calcium phosphate-based compound. The fluorescent protein derived from the cnidarian and the proteins other than the fluorescent protein derived from the cnidarian are specifically adsorbed to such an adsorbent 3 with adsorbability (supported power) which is inherently possessed by them. Therefore, the fluorescent protein and the proteins, that is, the contaminating proteins are separated from each other and purified based on a difference between the adsorbability of the fluorescent protein to the adsorbent 3 and the adsorbability of the proteins to the adsorbent 3.

Examples of the calcium phosphate-based compound include, but are not limited thereto, hydroxyapatite (Ca₁₀(PO₄)₆(OH)₂), TCP (Ca₃(PO₄)₂), Ca₂P₂O₇, Ca(PO₃)₂, DCPD (CaHPO₄.2H₂O), Ca₄O(PO₄)₂, materials in which a part of these materials is substituted by the other atoms or the other atom groups, and the like. These calcium phosphate-based compounds can be used singly or in combination of two or more of them.

In this regard, it is to be noted that the fluorescent protein derived from the cnidarian is generally an acid protein containing a relatively large amount of an acidic amino acid as a constituent amino acid thereof.

The calcium phosphate-based compound includes a large number of calcium atoms in a crystal structure thereof. A Ca site, which is capable of charging positively, is formed in the crystal structure.

In the fluorescent protein derived from the cnidarian, ion bonds are formed between the Ca site included in the calcium phosphate-based compound and the acidic amino acid contained in the fluorescent protein derived from the cnidarian. Therefore, the fluorescent protein derived from the cnidarian can be firmly bonded (adsorbed) to the phosphate calcium-based compound as compared to the contaminating proteins.

If the adsorbent 3 of which at least the surface is constituted of the phosphate calcium-based compound is used, it is possible to easily and reliability separate the fluorescent protein derived from the cnidarian (acid protein) from the contaminating proteins by utilizing the difference between adsorbability of the fluorescent protein derived from the cnidarian to the adsorbent 3 and adsorbability of the contaminating proteins to the adsorbent 3.

Further, the adsorbent 3 of which at least the surface is constituted of the phosphate calcium-based compound makes it possible to obtain high strength. Therefore, it is possible to reliably prevent the adsorbent 3 from being easily deformed and destroyed under its own weight for a long period of time. That is, such an adsorbent 3 makes it possible to prevent the following phenomenon from occurring. The adsorbent 3 filled into the lower portion of the adsorbent filling space 20 of the adsorption apparatus 1 is destroyed, thereby clogging occurs thereto.

As a result, it is possible to reliably treat a large amount of the sample solution. In other words, it becomes possible to reliably separate a large amount of the fluorescent protein derived from the cnidarian from the sample solution.

Among these calcium phosphate-based compounds mentioned above, one containing the hydroxyapatite as a main component of the adsorbent 3 is preferred. In particular, the hydroxyapatite is substance similar to components of a living body. Therefore, when the fluorescent protein derived from the cnidarian is adsorbed to and separated (desorbed) from the adsorbent 3, it is possible to reliably prevent such a fluorescent protein from being altered (denatured). Further, if the salt concentration of the phosphate elution buffer as an eluate is changed, the method according to the present invention has an advantage that the fluorescent protein derived from the cnidarian is specifically desorbed from the adsorbent 3.

It is preferred that at least one part of the hydroxyl groups of hydroxyapatite is substituted by fluorine atoms of hydrogen fluoride molecules to obtain fluoroapatite. The fluorine atoms exist in a crystal structure of fluoroapatite. This makes it possible to reliably prevent calcium atoms (calcium ions) from being separated or eliminated from fluoroapatite. Further, an adsorbent 3 of which at least the surface is constituted of the fluoroapatite makes it possible to further improve strength of the adsorbent 3.

Hereinafter, hydroxyapatite and fluoroapatite are collectively referred to as “apatite”.

In the meantime, in a fluorescent protein derived from Aequorea coerulescens belonging to the cnidarian, a chromophore (fluorophore) is formed by bonding three amino acids, namely, serine, tyrosine, and glycine together. As shown in the following formula (1), the chromophore has a structure in which two nitrogen atoms are adjacent each other. Therefore, if metal ions (calcium ions) are close to the structure (two nitrogen atoms) of the chromophore, a chelate bond is formed between the metal ions and the chromophore. It is a fear that fluorescent property of the fluorescent protein derived from Aequorea coerulescens is changed due to the chelate bond.

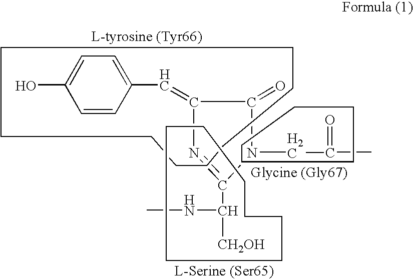

Therefore, there is necessity to firmly support the metal atoms to the adsorbent 3. However, the adsorbent 3 of which at least the vicinity of the surface is constituted of fluoroapatite makes it possible to reliably prevent the calcium ions from being eluted to the phosphate elution buffer which is used as an eluate or the sample solution. As a result, it is possible to reliably prevent fluorescent property of the separated fluorescent protein derived from Aequorea coerulescens from being changed due to the calcium ions.

In this regard, a method of producing fluoroapatite described above will be described later in detail.

Further, as shown in FIG. 1, the adsorbent 3 preferably has a particulate (granular) shape, but may have another shape such as a pellet (small block)-like shape or a block-like shape (e.g., a porous body in which adjacent pores communicate with each other or a honeycomb shape). By forming the adsorbent 3 having the particulate shape, it is possible to increase its surface area, and thereby improving separate characteristics of the fluorescent protein derived from the cnidarian to the adsorbent 3.

An average particle size of the adsorbent 3 is not particularly limited, but is preferably in the range of about 0.5 to 150 μm, and more preferably in the range of about 10 to 80 μm. By using the adsorbent 3 having such an average particle size, it is possible to reliably prevent clogging of the filter member 5 while a sufficient surface area of the adsorbent 3 is ensured.

It is to be noted that the adsorbent 3 may be entirely constituted of the calcium phosphate-based compound. Alternatively, the adsorbent 3 may be formed by coating the surface of a carrier (base) with the calcium phosphate-based compound. It is preferred that the adsorbent 3 may be entirely constituted of the calcium phosphate-based compound. This makes it possible to further improve strength of the adsorbent 3, thereby obtaining a suitable column to be used in separating a large amount of the fluorescent protein derived from the cnidarian.

The adsorbent 3 entirely constituted of the calcium phosphate-based compound can be obtained as follows. Phosphate calcium-based compound particles (primary particles) is obtained by using a wet synthesis method or a dry synthesis method, a slurry containing such phosphate calcium-based compound particles is prepared, and then the slurry is dried or granulated to obtain dried particles. Thereafter, the dried particles are sintered to obtain the adsorbent 3 entirely constituted of the calcium phosphate-based compound.

On the other hand, the adsorbent 3 formed by coating the surface of the carrier with the calcium phosphate-based compound can be obtained by using a method that the dried particles are collided (hybridized) with the carrier constituted of resins or the like.

In the case where almost the entire of the adsorbent filling space 20 is filled with the adsorbent 3 as in case of this embodiment, the adsorbent 3 preferably has substantially the same composition at every point in the adsorbent filling space 20. This makes it possible to allow the adsorption apparatus 1 to have a particularly excellent ability to separate (purify) the fluorescent protein derived from the cnidarian.

In this regard, it is to be noted that the adsorbent filling space 20 may be partially filled with the adsorbent 3 (e.g., a part of the adsorbent filling space 20 located on its one side where the inlet pipe 24 is provided may be filled with the adsorbent 3). In this case, the remaining part of the adsorbent filling space 20 may be filled with another adsorbent.

Hereinbelow, a method of separating the fluorescent protein derived from the cnidarian using the adsorption apparatus 1 described above (i.e., a separation method according to the present invention) will be described.

(1) Preparation Step

First, a plurality of proteins containing a fluorescent protein derived from a cnidarian is extracted from a sample to prepare a sample solution.

Examples of the sample to be used for extracting the fluorescent protein derived from the cnidarian include cnidarian such as Aequorea victoria and Obelia which belong to class Hydrozoa, and Porifera and Renilla which belong to class Anthozoa.

Examples of the other samples to be used for extracting the fluorescent protein derived from the cnidarian include: a mammal such as Ovis aries, Leporidae and Gallus gallus domesticus; an insect such as silkworm; an animal cell such as CHO cell derived from Chinese hamster ovary cell; materials secreted from a microbe such as coli bacteria; cytoplasmic components thereof; and the like. In these other samples, a nucleic acid including a gene corresponding to the fluorescent protein derived from cnidarian is transferred to nucleic acids thereof.

Among these samples mentioned above, the other samples are preferable. Such the other samples can produce a large amount of the fluorescent protein derived from the cnidarian (produced protein). Therefore, after the fluorescent protein derived from the cnidarian is extracted from the other samples to a sample solution, the fluorescent protein is separated from the sample solution and purified. This makes it possible to reliably and easily obtain the large amount of the fluorescent protein derived from the cnidarian.

Specifically, the nucleic acid including the gene corresponding to the fluorescent protein derived from cnidarian is transferred to a nucleic acid of the silkworm to produce the cocoon. Therefore, the cocoon produced by the silkworm is preferable as the sample.

If the cocoon produced by silkworm is used as the sample, it is possible to an extraction solution, in which the fluorescent protein derived from the cnidarian is extracted (dissolved), with relatively easy operations in that the cocoon (threads thereof) is dipped into a neutral buffer such as water and a normal saline. As a result, the extraction solution can be used as the sample solution which is used in the separation method according to the present invention.

It is difficult for the fluorescent protein derived from the cnidarian, which is produced by the silkworm, to be modified by sugar chains. Therefore, it is possible to relatively and easily obtain a fluorescent protein having a simple chemical structure (non-modified type protein). This makes it possible to prevent adsorbability of the fluorescent protein to the adsorbent 3 described above from being changed. That is to say, the separation method according to the present invention is optimally used for separating the fluorescent protein derived from the cnidarian.

If the nuclear acid including the gene corresponding to the fluorescent protein derived from the cnidarian is transferred to a nucleic acid of the silkworm to obtain a cocoon, and then the fluorescent protein derived from the cnidarian is separated from the cocoon produced by the silkworm with the separation method according to the present invention, it becomes possible to produce a pure fluorescent protein derived from the cnidarian on a commercial basis.

(2) Supplying Step

Next, the prepared sample solution is supplied to the adsorbent filling space 20 through the inlet pipe 24 and the filter member 4 to be in contact with the adsorbent 3 and to pass through the column 2 (adsorbent filling space 20).

The fluorescent protein derived from the cnidarian has high adsorbability to the adsorbent 3. Further, a part of the contaminating proteins other than the fluorescent protein derived from the cnidarian has relatively high adsorbability to the adsorbent 3. Therefore, the fluorescent protein derived from the cnidarian and contaminating proteins having the relatively high adsorbability are carried on the adsorbent 3 in the adsorbent filling space 20. The contaminating proteins having low adsorbability to the adsorbent 3 and foreign substances other than the contaminating proteins and the fluorescent protein derived from the cnidarian is discharged out of the column 2 through the filter member 5 and the outlet pipe 25.

(3) Fractionation Step

Next, a phosphate elution buffer as an eluate is supplied into the adsorbent filling space 20 (column 2) through the inlet pipe 24 and the filter member 4 to elute the fluorescent protein derived from the cnidarian, and thereby an eluant (eluate) containing the phosphate elution buffer and the fluorescent protein derived from the cnidarian can be obtained. Thereafter, the eluant discharged out of the column 2 through the outlet pipe 25 and the filler member 5 is fractionated (collected) into a portion of the phosphate elution buffer containing the fluorescent protein and other portions thereof to obtain fractions corresponding to the phosphate elution buffer having a predetermined amount of the eluant.

In this way, the fluorescent protein derived from the cnidarian and the contaminating proteins, which are adsorbed to the adsorbent 3, are collected (separated from each other) to the fractions in which they are eluted depending on the difference between absorbability of the fluorescent protein derived from the cnidarian to the adsorbent 3 and absorbability of the contaminating proteins to the adsorbent 3.

Examples of the phosphate elution buffer include sodium phosphate, potassium phosphate, lithium phosphate and the like.

A pH of the phosphate elution buffer is not particularly limited, but is preferably in the range of about 6 to 8, and more preferably in the range of about 6.5 to 7.5. This makes it possible to prevent the fluorescent protein derived from the cnidarian to be separated from being altered, thereby preventing fluorescent property from being changed. In addition, it is also possible to reliably prevent the adsorbent 3 from being altered (dissolved), thereby preventing separation property of the adsorbent 3 from being changed in the adsorption apparatus 1.

A temperature of the phosphate elution buffer is not particularly limited either, but is preferably in the range of about 30 to 50° C., and more preferably in the range of about 35 to 45° C. This makes it possible to prevent the fluorescent protein derived from the cnidarian to be separated from being altered.

By using the phosphate elution buffer of which pH and temperature fall within the above noted ranges, it is possible to improve a collection rate of a target fluorescent protein derived from the cnidarian.

A salt concentration of the phosphate elution buffer is preferably 500 mM or less, and more preferably 400 mM or less. The separation of the fluorescent protein derived from the cnidarian by using the phosphate elution buffer having such a salt concentration makes it possible to prevent adverse affects from occurring to the fluorescent protein derived from the cnidarian due to existence of the metal ions in the phosphate elution buffer.

The salt concentration of the phosphate elution buffer is preferably in the range of about 1 to 400 mM. Further, it is preferred that the salt concentration of the phosphate elution buffer is changed in a continuous manner or a stepwise manner when a separate operation of the fluorescent protein derived from the cnidarian. This makes it possible to efficiently improve the separate operation of the fluorescent protein derived from the cnidarian.

A flow rate of the phosphate elution buffer to flow in the adsorbent filling space 20 is preferably in the range of about 0.1 to 10 mL/min, and more preferably in the range of about 1 to 5 mL/min. By separating the fluorescent protein derived from the cnidarian from the sample solution at such a flow rate, it is possible to reliably separate a target fluorescent protein derived from the cnidarian from the sample solution without long time to be needed to the separation operation. That is to say, it is possible to obtain a large amount of the fluorescent derived from the cnidarian, or to obtain the fluorescent derived from the cnidarian having high purity.

By the operations as described above, the fluorescent protein derived from the cnidarian is collected to a predetermined of fractions.

In various kinds of amino acids, a basic amino acid such as histidine, lysine, or arginine has high affinity to the metal ions. Therefore, if the altered body of the fluorescent protein derived from the cnidarian is obtained by adding at least one of histidine, lysine, and arginine to a fluorescent protein derived from a natural cnidarian, the fluorescent protein derived from the natural cnidarian can be collected with more high collection rate (yield) by using the separation method according to the present invention.

Further, the basic amino acid has high affinity to a zinc atom (zinc ion), a nickel atom (nickel ion), cobalt atom (cobalt ion), and copper atom (copper ion). Therefore, at least one part of the calcium atoms of apatite such as hydroxyapatite and fluoroapatite described above may be substituted by these atoms (ions). This makes it possible to improve affinity of the fluorescent protein derived from cnidarian to the adsorbent 3.

Examples of a method of substituting the at least one part of the calcium atoms of apatite by the atoms (ions) include a method of bringing a liquid containing a halide, a hydroxide, a sulfated material, a carbonated material, and the like, in which the atoms contained, into contact with the apatite. According to such a method, the atoms can be substituted to the calcium atoms with relatively ease.

In the aforementioned description, the fluorescent protein derived from the cnidarian (or altered body thereof) has described as one example of a fluorescent protein. However, the separation method according to the present invention is also capable of separating a fluorescent protein contained in fish such as Anguilla japonica with ease and high purity.

In the meantime, fluoroapatite as described above may be produced by using any kind of method. It is preferred that fluoroapatite is produced by using the following methods (I) or (II).

The method (I) is a method of substituting at least one part of hydroxyl groups of hydroxyapatite by fluorine atoms of hydrogen fluoride molecules. Such a method is carried out by reacting hydroxyapatite and the hydrogen fluoride molecules in a mixture which is obtained by mixing a slurry containing hydroxyapatite and a hydrogen fluoride-containing solution containing the hydrogen fluoride molecules to thereby obtain fluoroapatite.

The method (II) is carried out as follows. A first liquid containing a calcium-based compound containing calcium, a second liquid containing the hydrogen fluoride molecules and a third liquid containing phosphoric acid are prepared, respectively. Thereafter, the first liquid, the second liquid and the third liquid are mixed to obtain first mixture. Then the calcium-based compound, the hydrogen fluoride molecules and phosphoric acid are reacted in the first mixture to thereby obtain fluoroapatite.

In a conventional method of synthesizing fluoroapatite, fluoroapatite is synthesized by adding ammonium hydrogen fluoride as a fluorine source to a slurry containing hydroxyapatite. However, according to these methods (I) and (II), since the hydrogen fluoride molecules are used as the fluorine source, it is possible to obtain fluoroapatite in which no impurity is contained or an impurity is contained within a very low level.

Therefore, it is possible to obtain fluoroapatite having high crystallinity. Further, it is possible to improve acid resistance of the produced fluoroapatite due to the high crystallinity. Therefore, the adsorbent 3 constituted of such fluoroapatite can be used for separating a fluorescent protein from a sample solution with an eluate having a relatively low pH. In this case, the separation process can be carried out without dissolution of the adsorbent 3. Therefore, it becomes possible to reliably separate such a fluorescent protein from the sample solution.

Furthermore, since the obtained fluoroapatite has a large specific surface area, the use of the adsorbent 3 constituted of such fluoroapatite makes it possible to improve a collection rate of the fluorescent protein.

Hereinafter, a description will be made on the methods (I) and (II).

<Method I>

A1: First, a slurry containing hydroxyapatite is prepared.

Hereinbelow, a method of preparing hydroxyapatite primary particles and a slurry in which aggregates of the hydroxyapatite primary particles are dispersed will be described.

The hydroxyapatite primary particles can be obtained by various synthesis methods, but are preferably synthesized by a wet synthesis method in which at least one of a calcium source (calcium compound) and a phosphoric acid source (phosphoric acid compound) is used in the form of a solution.

Further, the thus produced hydroxyapatite primary particles are small in size, and have therefore very highly reactive with hydrogen fluoride.

Examples of the calcium source to be used in the wet synthesis of the present invention include calcium hydroxide, calcium oxide, calcium nitrate and the like. Examples of the phosphoric acid source to be used in the wet synthesis of the present invention include phosphoric acid, ammonium phosphate and the like. Among them, one mainly containing the calcium hydroxide or the calcium oxide is particularly preferred as the calcium source, and one mainly containing the phosphoric acid is particularly preferred as the phosphoric acid source.

More specifically, such hydroxyapatite primary particles and slurry can be obtained by dropping a phosphoric acid (H₃PO₄) solution into a suspension of calcium hydroxide (Ca(OH)₂) or calcium oxide (CaO) contained in a container and mixing them by stirring.

An amount of the hydroxyapatite primary particles contained in the slurry is preferably in the range of about 1 to 20 wt %, and more preferably in the range of about 5 to 12 wt %.

A2: On the other hand, a solution containing hydrogen fluoride is prepared separately from the slurry containing the hydroxyapatite.

A solvent for dissolving hydrogen fluoride is not particularly limited, and any solvent can be used as long as it does not inhibit a reaction between hydroxyapatite and hydrogen fluoride.

Examples of such a solvent include water, an alcohol such as methanol and ethanol, and the like. These solvents may be used in combination of two or more of them. However, among them, water is particularly preferred.

An amount of the hydrogen fluoride contained in the hydrogen fluoride-containing solution is preferably in the range of about 1 to 60 wt %, and more preferably in the range of about 2.5 to 10 wt %.

A3: Next, the prepared slurry and the prepared hydrogen fluoride-containing solution are mixed together to react the hydroxyapatite primary particles with the hydrogen fluoride in the slurry (reaction liquid) containing the hydrogen fluoride-containing solution to obtain fluoroapatite primary particles.

More specifically, as shown in the following formula, by bringing the hydroxyapatite primary particles into contact with hydrogen fluoride, it is possible to substitute at least one part of the hydroxyl groups of hydroxyapatite by the fluorine atoms of hydrogen fluoride molecules to convert the hydroxyapatite into fluoroapatite and thereby to obtain the fluoroapatite primary particles.

Ca₁₀(PO₄)₆(OH)₂→Ca₁₀(PO₄)₆(OH)₂₋₂ₓF₂ₓ

(wherein 0<x≦1)

As described above, by reacting the hydroxyapatite primary particles with hydrogen fluoride in the slurry containing the hydroxyapatite primary particles, it is possible to easily produce the fluoroapatite primary particles.

Further, since the hydroxyl groups of hydroxyapatite are substituted by the fluorine atoms of the hydrogen fluoride molecules during the stage of the hydroxyapatite primary particles, the obtained fluoroapatite primary particles have a particularly high rate of substitution of the hydroxyl groups by the fluorine atoms.

Further, since hydrogen fluoride (HF) is used as a fluorine source, no by-product is formed or an amount of a formed by-product is extremely small as compared to a case where ammonium hydrogen fluoride (NH₄F), lithium fluoride (LiF), sodium fluoride (NaF), potassium fluoride (KF), magnesium fluoride (MgF₂), calcium fluoride (CaF₂), or the like is used as the fluorine source.

More specifically, the impurity content of fluoroapatite is preferably as small as possible. For example, it is preferably 300 ppm or less, and more preferably 100 ppm or less. This makes it possible to prevent or suppress the fluorine atoms from being eliminated from fluoroapatite due to their low impurity content, thereby improving acid resistance of the fluoroapatite primary particles.

According to the present invention, by adjusting the reaction conditions (e.g., pH, temperature, time) of the reaction between the hydroxyapatite (primary particles) and hydrogen fluoride, it is possible to allow the impurity content contained in the fluoroapatite primary particles to reliably fall within the above range. Further, it is possible to allow a concentration of a fluorine ion contained in a supernatant to reliably fall within a predetermined range.

Particularly, according to the present invention, the pH of the slurry is adjusted to fall within the range of 2.5 to 5 by mixing the hydrogen fluoride-containing solution with the slurry, and in this state, the hydroxyapatite (primary particles) reacts with hydrogen fluoride. This makes it possible to allow the concentration of the fluorine ion and the impurity content to reliably fall within the above range. In this regard, it is to be noted that in this specification, the pH of the slurry means a pH value at the time when an entire amount of the hydrogen fluoride-containing solution is mixed with the slurry.

If the pH of the slurry is adjusted to less than 2.5, there is a tendency that hydroxyapatite itself dissolves, and therefore it becomes difficult to convert hydroxyapatite into fluoroapatite to obtain fluoroapatite primary particles. Further, in this case, there is also a problem that constituent materials of a device for use in mixing the hydroxyapatite primary particles with the hydrogen fluoride-containing solution are eluted into the slurry so that low-purity fluoroapatite primary particles are obtained. Furthermore, it is technically very difficult to adjust the pH of the slurry to a low value less than 2.5 using the hydrogen fluoride-containing solution.

On the other hand, in order to adjust the pH of the slurry to more than 5 using the hydrogen fluoride-containing solution, a large amount of water has to be added to the slurry. In this case, a total amount of the slurry becomes extremely large, and as a result, the yield of the fluoroapatite primary particles based on the total amount of the slurry is lowered. This is industrially disadvantageous.

In contrast to the above two cases, in a case where the pH of the slurry is adjusted to fall within the range of 2.5 to 5, the fluoroapatite (primary particles) produced by the reaction once tends to dissolve and is then recrystallized. Therefore, the fluoroapatite primary particles having high crystallinity can be obtained.

The slurry and the hydrogen fluoride-containing solution may be mixed together at one time, but they are preferably mixed by adding (dropping) the hydrogen fluoride-containing solution into the slurry drop by drop.

A rate of dropping the hydrogen fluoride-containing solution into the slurry is preferably in the range of about 1 to 100 L/hr, and more preferably in the range of about 3 to 100 L/hr.

Further, the reaction between the hydroxyapatite primary particles and hydrogen fluoride is preferably carried out while the slurry is stirred. By stirring the slurry, it is possible to bring the hydroxyapatite primary particles into uniform contact with hydrogen fluoride and thereby to allow the reaction between the hydroxyapatite primary particles and hydrogen fluoride to efficiently proceed. In addition, it is also possible to obtain fluoroapatite primary particles more uniform in the rate of substitution of the hydroxyl groups of hydroxyapatite by the fluorine atoms of the hydrogen fluoride molecules. By using such fluoroapatite primary particles, it is possible to produce, for example, an adsorbent (dried particles or sintered particles) having less characteristic variations and high reliability.

In this case, power for stirring the slurry is preferably in the range of about 0.1 to 3 W, and more preferably in the range of about 0.5 to 1.8 W per 1 liter of the slurry.

An amount of hydrogen fluoride to be mixed is determined so that an amount of the fluorine atoms becomes preferably in the range of about 0.65 to 1.25 times, and more preferably in the range of about 0.75 to 1.15 times with respect to an amount of the hydroxyl groups of hydroxyapatite.

A temperature of the reaction between the hydroxyapatite primary particles and hydrogen fluoride is not particularly limited, but is preferably in the range of about 5 to 50° C., and more preferably in the range of about 20 to 40° C.

In this case, hydrogen fluoride is preferably dropped (added) into (to) the slurry containing the hydroxyapatite primary particles for a length of time from about 30 minutes to 16 hours, and more preferably for a length of time from about 1 to 8 hours.

Method II

B1: First, a first liquid containing a calcium-based compound containing calcium as a calcium source is prepared.

Examples of the calcium-based compound (calcium source) to be contained in the first liquid include, but not limited thereto, calcium hydroxide, calcium oxide, calcium nitrate and the like. These compounds may be used singly or in combination of two or more of them. Among them, calcium hydroxide is particularly preferred as the calcium source.

A solution or suspension containing the calcium-based compound as the calcium source can be used as the first liquid. In the case where the calcium-based compound is calcium hydroxide, a calcium hydroxide suspension in which the calcium hydroxide is suspended in water is used preferably.

An amount of the calcium-based compound as the calcium source contained in the first liquid is preferably in the range of about 1 to 20 wt %, and more preferably in the range of about 5 to 12 wt %.

B2: Next, a second liquid containing hydrogen fluoride (hydrogen fluoride-containing solution) is prepared.

A solvent for dissolving hydrogen fluoride is not particularly limited, and any solvent can be used as long as it does not inhibit a reaction to be carried out in the step B5 which will be described later.

Examples of such a solvent include water, an alcohol such as methanol and ethanol, and the like. These solvents may be used in combination of two or more of them. However, among them, water is particularly preferred.

B3: Next, a third liquid containing phosphoric acid (phosphoric acid-containing solution) is prepared.

A solvent for dissolving phosphoric acid is not particularly limited, and any solvent can be used as long as it does not inhibit the reaction to be carried out in the step B5 which will be described later. The same solvent can be used as the solvent for dissolving hydrogen fluoride in the step B2 described above.

It is to be noted that both the solvent for dissolving hydrogen fluoride and the solvent for dissolving phosphoric acid are preferably the same kind of solvent or the same solvent.

A first mixture is obtained by mixing the first, second and third liquids prepared as described above. The mixing order thereof is not limited as long as the calcium-based compound, hydrogen fluoride and phosphoric acid can be simultaneously existed in the first mixture in the step S5 described later. However, it is preferred that after the second liquid is mixed with the third liquid to obtain a second mixture, and then the second mixture is added to the first liquid to obtain the first mixture.

By mixing the first, second and third liquids in this order, the second liquid and the third liquid can be uniformly mixed with the first liquid. Further, the hydroxyl groups of hydroxyapatite can be uniformly substituted by the fluorine atoms of the hydrogen fluoride molecules. Furthermore, it is possible to reliably prevent or suppress a by-product such as calcium fluoride from being produced.

In this regard, it is to be noted that examples of a method of obtaining the first mixture other than the method described above include: a method of substantially simultaneously adding the second liquid and the third liquid to the first liquid; a method of substantially simultaneously adding the first liquid and the third liquid to the second liquid; and a method of substantially simultaneously adding the first liquid and the second liquid to the third liquid.

Hereinafter, a description will be made, as a representative, with respect to the case where after the second mixture is prepared, the second mixture is mixed with the first liquid to obtain the first mixture, thereby producing fluoroapatite.

B4 Next, the second liquid and the third liquid, which have been prepared in the steps B2 and B3, respectively, are mixed to each other to obtain the second mixture.

An amount of hydrogen fluoride contained in the second mixture is preferably in the range of about 0.5 to 60 wt %, and more preferably in the range of about 1.0 to 10 wt %.

An amount of phosphoric acid contained in the second mixture is preferably in the range of about 1.0 to 90 wt %, and more preferably in the range of about 5.0 to 20 wt %.

The amount of phosphoric acid contained in the second mixture is preferably in the range of about 2.0 to 4.5 times in a mol amount, and more preferably in the range of about 2.8 to 4.0 times with respect to hydrogen fluoride contained in the second mixture at the mol amount.

B5: Next, the first liquid (solution containing calcium-based compound) prepared in the step B1 described above is mixed with the second mixture obtained in the step B4 described above to obtain the first mixture. Then, the calcium-based compound as a calcium source is reacted with hydrogen fluoride and phosphoric acid in the first mixture to thereby obtain fluoroapatite primary particles.

More specifically, in the case where calcium hydroxide is used as the calcium source, by bringing calcium hydroxide into contact with hydrogen fluoride and phosphoric acid, it is possible to obtain fluoroapatite primary particles as shown in the following formula.

10Ca(OH)₂+6H₃(PO₄)+2HF→Ca₁₀(PO₄)₆(OH)₂₋₂ₓF₂ₓ+18H₂O+2(H₂O)x+2HF₁₋ₓ

(wherein 0<x≦1)

As described above, the fluoroapatite primary particles can be reliably produced by bringing hydrogen fluoride and phosphoric acid into contact with the calcium-based compound (calcium hydroxide) as the calcium source, and then reacting hydrogen fluoride, phosphoric acid and the calcium-based compound with simple handling that the first liquid is mixed with the second mixture.

Fluoroapatite produced by the reaction as shown in the above formula has a large specific surface area.

As shown in the above formula, it is supposed that fluoroapatite is produced by substituting the hydroxyl groups of hydroxyapatite by the fluorine atoms of the hydrogen fluoride molecules simultaneously with producing hydroxyapatite primary particles. Therefore, it is possible to obtain a high ratio of substituting the hydroxyl groups of hydroxyapatite by the fluorine atoms of the hydrogen fluoride molecules.

Further, since hydrogen fluoride (HF) is used as a fluorine source in the present invention, no by-product is formed or an amount of a by-product is extremely small as compared to a case where ammonium fluoride (NH₄F), lithium fluoride (LiF), sodium fluoride (NaF), potassium fluoride (KF), magnesium fluoride (MgF₂), calcium fluoride (CaF₂), or the like is used as the fluorine source. Therefore, the amount of the by-product (impurity) contained in fluoroapatite primary particles can be made small so that acid resistance of the fluoroapatite primary particles is improved. It is to be noted that the term “impurity” used herein means ammonia, lithium, calcium fluoride or the like which is derived from a raw material of fluoroapatite.

More specifically, the impurity content of fluoroapatite is preferably as small as possible. For example, it is preferably 300 ppm or less, and more preferably 100 ppm or less. This makes it possible to further improve acid resistance of the fluoroapatite primary particles due to their low impurity content.

According to the present invention, by adjusting the conditions (e.g., pH, temperature, time) of the reaction among the calcium-based compound (calcium source), hydrogen fluoride and phosphoric acid, it is possible to allow the impurity content contained in the fluoroapatite primary particles to fall within the above range.

The first liquid and the second mixture may be mixed together at one time to obtain the first mixture, but they are preferably mixed by adding (dropping) the second mixture into the first liquid drop by drop. By dropping the second mixture into the first liquid, it is possible to relatively easily react the calcium-based compound, hydrogen fluoride and phosphoric acid.

It is possible to more easily and reliably adjust a pH of the second mixture to a value within an appropriate range. For these reasons, decomposition or dissolution of the produced fluoroapatite can be prevented. As a result, it is possible to obtain fluoroapatite (fluoroapatite primary particles) having high purity and a large specific surface area in a high yield.

A rate of dropping the second mixture into the first liquid is preferably in the range of about 1 to 100 L/hr, and more preferably in the range of about 10 to 100 L/hr. By mixing (adding) the second mixture with (to) the first liquid at such a dropping rate, it is possible to react the calcium-based compound, hydrogen fluoride and phosphoric acid under milder conditions.

Further, the reaction among the calcium-based compound, hydrogen fluoride and phosphoric acid is preferably carried out while the first mixture is stirred. By stirring the first mixture, it is possible to bring the calcium-based compound into uniformly contact with hydrogen fluoride and phosphoric acid and thereby to allow the reaction among the calcium-based compound, hydrogen fluoride and phosphoric acid to efficiently proceed. In addition, the hydroxyl groups of hydroxyapatite are uniformly substituted by the fluorine atoms of the hydrogen fluoride molecules. By using such fluoroapatite primary particles, it is possible to produce, for example, an adsorbent (dried particles or sintered particles) 3 having less characteristic variations and high reliability.

In this case, power for stirring the first mixture (slurry) is preferably in the range of about 0.5 to 3 W, and more preferably in the range of about 0.9 to 1.8 W per 1 liter of the slurry.

A temperature of the reaction among the calcium-based compound as the calcium source, hydrogen fluoride and phosphoric acid is not particularly limited, but is preferably in the range of about 5 to 50° C., and more preferably in the range of about 20 to 40° C.

Although the separation method according to the present invention has been described above with reference to preferred embodiments thereof, the present invention is not limited thereto. For example, the separation method according to the present invention may further include one or more steps for any purpose.

### EXAMPLES

Hereinbelow, the present invention will be described with reference to specific examples.

### Example 1

-1- First, a silkworm cocoon (threads of cocoon) containing a recombinant GFP (fluorescent protein derived from Aequorea Victoria) was grinded to a powder state by using a grinder to obtain a powder of the silkworm cocoon.

In this regard, the recombinant GFP was an altered body in which six histidines were bonded to the fluorescent protein (GFP) derived from natural Aequorea Victoria. The silkworm cocoon containing the recombinant GFP was obtained from NEO SILK Co., Ltd.

-2- Next, 50 mM tris hydrochloric buffer solution (pH 7.5) containing 150 mM NaCl was added to the powder of 120 mg to obtain a mixture, thereafter the obtained mixture was stirred. In this regard, it is to be noted that the stirring conditions were set so that a stirring speed was 30 rpm, a temperature of the tris hydrochloric buffer solution was 4° C., and a stirring time was 48 hours.

-3- Next, the stirred mixture was subjected to a centrifugal separation treatment (15000 rpm, for 5 minutes at a temperature of 4° C.) to obtain a supernatant, and then the supernatant was concentrated by using an ultrafiltration method. Thus concentrated supernatant was used as a sample solution which contained the recombinant GFP and contaminating proteins derived from the silkworm cocoon.

-4- Next, 50 μL of the sample solution was supplied (applied) into an adsorbent filling space of a adsorption apparatus to adsorb the recombinant GFP and contaminating proteins to an adsorbent. Then, an eluate A was supplied into the adsorbent filling space for 5 minutes at a flow rate of 1 mL/min. Next, a mixture of the eluate A and an eluate B was supplied into the adsorbent filling space for 15 minutes at a flow rate of 1 mL/min so that an amount ratio between the eluate A and the eluate B was continuously changed in the range of 0 to 100%. Thereafter, the eluate B was supplied into the adsorbent filling space for 5 minutes at a flow rate of 1 mL/min. By supply process as described above, the recombinant GFP and contaminating proteins were desorbed from the adsorbent to the eluate A, the mixture, or the eluate B to thereby obtain an eluant containing the recombinant GFP and/or the contaminating proteins. Then, the eluant containing the recombinant GFP and/or the contaminating proteins were/was discharged from the adsorbent filling space to out of the adsorption apparatus. The discharged eluant was fractionated in vessels by 2 mL.

In this regard, it is to be noted that a column (size 4 mm×100 mm) in which 0.9 g of hydroxyapatite beads (“CHT Typell”, of which average diameter was 40 μm, was produced by Pentax (HOYA corporation).) as the adsorbent was filled into the adsorbent filling space was used in the adsorption apparatus.

Further, it is to be noted that 1 mM phosphate elution buffer (pH 6.8) was used as the eluate A and 400 mM phosphate elution buffer (pH 6.8) was used as the eluate B.

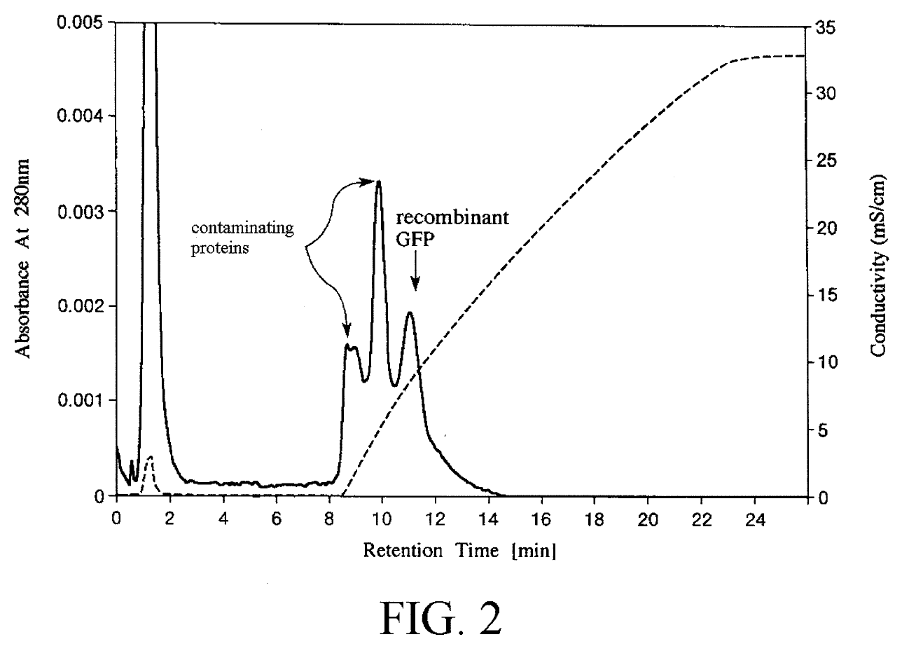

As a result, the fluorescent protein derived from Aequorea Victoria could be separated from the contaminating proteins derived from the silk cocoon which were contained in the sample solution. This result was shown as peaks in which one peak in about 11 minutes of the retention time in FIG. 2 represented a peak of the recombinant GFP and the other peaks in the range of about 8 to 10 minutes of the retention time in FIG. 2 represented peaks of the contaminating proteins. That is to say, the fluorescent protein derived from Aequorea Victoria could be collected in fractions (the vessels) containing the eluant which was discharged from the adsorbent filling space to out of the adsorption apparatus in the range of about 10 to 12 minutes.

Likewise, above processes were carried out 50 times repeatedly. These results were the same as the above results.

### Comparative Example

According to a method described in M. Tomita et al., Transgenic Res., 16, 449-465, 2007., a recombinant GFP which was the same as that used in the Example 1 was separated from contaminating proteins derived from a silkworm cocoon by using a Ni-affinity column.

As a result, the separation and the collection of the recombinant GFP was possible. However, clogging occurred to the Ni-affinity column at a time in which the same processes as those in the Example 1 were repeated 30 times.

### Example 2

A natural GFP extracted from Aequorea Victoria was separated from contaminating proteins derived from a silkworm cocoon and collected in fractions as same manner in the Example 1. The results were the same as that of the Example 1. Further, according to a method described in US-A-2008-0301823, a natural GFP was produced in threads constituting a silkworm cocoon, and then the natural GFP was separated from contaminating proteins derived from the silkworm cocoon and collected in fractions as same manner in the Example 1. The results were the same as that of the Example 1. In this regard, a number of histidine contained in the natural GFP was smaller than that contained in the recombinant GFP. Therefore, there was a tendency that the retention time of the natural GFP in an absorbance curve was slightly earlier than that of the recombinant GFP due to the number of histidine contained therein.

Furthermore, the natural GFP were separated from the contaminating proteins derived from the silkworm cocoon and collected in the fractions as the same manner in the Example 1 by using the fluoroapatite beads produced as described above as an adsorbent. The results were the same as that of the Example 1. In this regard, there was a tendency that the use of such an adsorbent makes it possible to improve a number of the separation operation to be carried out repeatedly.

Furthermore, at least one of calcium atoms of hydroxylapatite was substituted by at least one of zinc atoms (zinc ions), nickel atoms (nickel ions), cobalt atoms (cobalt ions), and copper atoms (copper ions) to obtain hydroxylapatite beads as the adsorbent. A natural GFP were separated from contaminating proteins derived from a silkworm cocoon and collected in fractions as the same manner in the Example 1 by using such hydroxylapatate beads. The results were the same as that of Example 1.

In this regard, there was a tendency that each of the retention times of the natural GFP and the contaminating proteins separated by using such hydroxylapatate beads as the adsorbent became later than those of the natural GFP and the contaminating proteins separated by using the adsorbent in the Example 1 due to the improved affinity between the natural GFP and the adsorbent. This tendency was shown conspicuously in case of the use of the recombinant GFP.

Furthermore, a recombinant GFP were separated from contaminating proteins derived from a silkworm cocoon and collected in fractions as the same manner in the Example 1 except that the recombinant GFP was changed to a fluorescent protein derived from Anguilla japonica. The results were the same as that of Example 1.

Furthermore, it is also to be understood that the present disclosure relates to subject matter contained in Japanese Patent Application No. 2008-042209 (filed on Feb. 22, 2008) which is expressly incorporated herein by reference in its entirety.

Unless otherwise stated, a reference to a compound or component includes the compound or component by itself, as well as in combination with other compounds or components, such as mixtures of compounds.

As used herein, the singular forms, “a,” “an,” and “the” include the plural reference unless the context clearly dictates otherwise.

Except where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not to be considered as an attempt to limit the application of the doctrine of equivalents to the scope of the claims, each numerical parameter should be construed in light of the number of significant digits and ordinary rounding conventions.

Additionally, the recitation of numerical ranges within this specification is considered to be a disclosure of all numerical values and ranges within that range. For example, if a range is from about 1 to about 50, it is deemed to include, for example, 1, 7, 34, 46.1, 23.7, or any other value or range within the range.

What is claimed is:

1. A method of separating a fluorescent protein from a sample containing a plurality of proteins containing the fluorescent protein, the method comprising:
preparing a sample solution by adding the sample to a liquid;
preparing an adsorption apparatus having a filling space for filling an adsorbent having a surface, wherein at least the surface of the adsorbent is constituted of a calcium phosphate-based compound and at least a part of the filling space is filled with the adsorbent;
supplying the sample solution into the filling space of the adsorption apparatus so that the plurality of proteins are adsorbed by the adsorbent;
supplying a phosphate elution buffer for eluting the fluorescent protein contained in the plurality of proteins from the adsorbent into the filling space of the adsorption apparatus to thereby obtain an eluant containing the fluorescent protein; and
fractionating the eluant which is discharged from the filling space of the adsorption apparatus into a portion of the phosphate elution buffer containing the fluorescent protein and other portions thereof to thereby separate the fluorescent protein from the plurality of proteins.

2. The method as claimed in claim 1, wherein in the phosphate elution buffer supplying step, a pH of the phosphate elution buffer is in the range of 6 to 8.

3. The method as claimed in claim 1, wherein in the phosphate elution buffer supplying step, a temperature of the phosphate elution buffer is in the range of 30 to 50° C.

4. The method as claimed in claim 1, wherein in the phosphate elution buffer supplying step, a salt concentration of the phosphate elution buffer is 500 mM or lower.

5. The method as claimed in claim 1, wherein in the phosphate elution buffer supplying step and the eluant fractionating step, a flow rate of the phosphate elution buffer flowing in the filling space of the adsorption apparatus is in the range of 0.1 to 10 mL/min.

6. The method as claimed in claim 1, wherein the fluorescent protein is at least one of a fluorescent protein derived from a cnidarian and an altered body thereof.

7. The method as claimed in claim 6, wherein the altered body is obtained by adding at least one of histidine, lysine, and arginine to the fluorescent protein derived from the cnidarian.

8. The method as claimed in claim 6, wherein the fluorescent protein is expressed in threads constituting a silkworm cocoon by transferring a nucleic acid including a gene corresponding to the fluorescent protein to a nucleic acid of the silkworm.

9. The method as claimed in claim 1, wherein the calcium phosphate-based compound is constituted of hydroxyapatite as a main component thereof.

10. The method as claimed in claim 9, wherein the hydroxyapatite has hydroxyl groups, and the hydroxyapatite is reacted with hydrogen fluoride molecules having fluorine atoms to obtain a fluoroapatite, wherein at least one of the hydroxyl groups of the hydroxyapatite is substituted by the fluorine atoms of the hydrogen fluoride molecules.

11. The method as claimed in claim 10, wherein the fluoroapatite is produced by preparing a slurry containing the hydroxyapatite, preparing a hydrogen fluoride-containing solution containing the hydrogen fluoride molecules, mixing the slurry and the hydrogen fluoride-containing solution to obtain a mixture thereof, and reacting the hydroxyapatite contained in the slurry and the hydrogen fluoride molecules contained in the hydrogen fluoride-containing solution in the mixture to thereby substitute the at least one of the hydroxyl groups of the hydroxyapatite to the fluorine atoms of the hydrogen fluoride molecules.

12. The method as claimed in claim 11, wherein a pH of the mixture is in the range of 2.5 to 5.0.

13. The method as claimed in claim 10, wherein the fluoroapatite is produced by preparing a first liquid containing a calcium-based compound containing calcium, a second liquid containing the hydrogen fluoride and a third liquid containing phosphoric acid, respectively, and thereafter obtaining a first mixture by mixing the first liquid, the second liquid and the third liquid, and then reacting the calcium-based compound, the hydrogen fluoride and the phosphoric acid in the first mixture.

14. The method as claimed in claim 13, wherein the first mixture obtaining step is carried out by mixing the second liquid and the third liquid to obtain a second mixture and thereafter mixing the second mixture with the first liquid.
